(19) United States
(12) Patent Application Publication
(10) Pub. No.: US 2006/0094957 A1
(43) Pub. Date: May 4, 2006

(54) Marker and cut down guide assembly for human mammary duct procedures and method

(76) Inventors: Richard L. Mueller JR., Jackson, WY (US); James Kermode, Los Altos, CA (US)

Correspondence Address: OLSON & HIERL, LTD., 20 NORTH WACKER DRIVE, 36TH FLOOR, CHICAGO, IL, 60606, US

(21) Appl. No.: 10/978,988

(22) Filed: Nov. 1, 2004

(51) Int. Cl. A61B 6/00; A61N 5/00; A61B 10/00

(52) U.S. Cl. 600/431; 600/562; 600/3

(57) ABSTRACT

A biopsy assembly suitable for marking of a biopsy site and providing a cut down guide. The assembly includes a pliable filament having an indicator for marking the biopsy site and a stiffener guidable along the filament to provide a cut down guide.

### TECHNICAL FIELD OF THE INVENTION

The invention relates to an assembly for use as a biopsy site marker as well as a cut down guide for surgical procedures.

### BACKGROUND OF THE INVENTION

Breast cancer is one of the health threats most feared by women, and is the most common form of cancer in women. A key to treatment is early detection. For example, a mammogram is a procedure that has been used in hopes of early detection of breast cancer. One problem with mammography is that such an imaging technique can only find breast cancer once it has taken form. All too often, breast cancer is discovered at a stage that is too far advanced, when therapeutic options and survival rates are severely limited. Such methods and devices could significantly improve breast cancer survival. While breast cancer is most common among women, in rare instances the human male may also have occurrences of breast cancer.

Early detection is a key to cancer treatment. As such, more sensitive and reliable methods and devices are needed to detect cancerous, pre-cancerous, and other cancer markers of the breast at an early stage. For example, there are methods of detecting breast cancer which are based on the fact that a vast majority of instances of breast cancer begins in the lining of mammary ducts. Studies have shown that fluid within the mammary duct can contain high levels of breast cancer markers, and that an estimated 80%-90% of all breast cancers occur within the intraductal epithelium of the mammary glands.

Fluid within the breast ducts contains an assemblage and concentration of hormones, growth factors and other potential markers comparable to those secreted by, or acting upon, the surrounding cells of the alveolar-ductal system. Likewise, mammary fluid typically contains cells and cellular debris, or products that can also be used in cytological or immunological assays. One method for obtaining samples of cells from a mammary duct is ductal lavage. This method comprises the rinsing of a mammary duct with a saline solution or the like and collection of the solution along with any cells and cellular debris from the mammary duct.

Other methods of obtaining cellular or tissue samples from mammary ducts include brushing biopsies or targeted cutting operations. In brushing biopsy procedures, an introducer is guided through the nipple orifice and down a mammary duct. Typically the progress of the introducer is guided by an endoscope therein. When the distal end portion of the introducer is at the desired location, a brush is extended to scrape the intraductal epithelium. The loosened or dislodged tissue may then be collected for pathology, such as by suction. In targeted cutting operations a biopsy device, such as a cutter is guided through the introducer. For example, a biopsy device, such as shown in pending application U.S. Ser. No. 10/422,381, is utilized to excise a tissue sample by urging the desired mass through a window in a sheath and then actuating a cutter also disclosed within the sheath to excise the mass. The excised tissue is harvested and analyzed.

In the event that pathology reveals that a cellular or tissue sample so obtained warrants further examination, additional surgery or other procedures at the biopsy site may be conducted. Therefore, it is desired that the original biopsy site be marked so that such further examination of the site is facilitated. In order to mark the biopsy site, prior procedures have utilized the deposit of a radio-opaque marker at the biopsy site. Later investigations utilize an x-ray or ultrasound to view the marker to enable the practitioner to return to the site. A problem with such a marker, however, is that in mammary ducts there are numerous branches that make returning to the biopsy site through the duct system difficult.

Assuming that the biopsy site is marked or otherwise found, a further problem exists regarding how the physician reach the site for further procedures. As discussed, a radio-opaque marker is visible through use of an x-ray or ultrasound, however, such equipment is not amenable to use during surgery. Feeling for the desired tissue mass is also not possible since, as described, the biopsy site involves masses that are very small or are visually and tactilely indistinguishable from surrounding tissue.

In order to avoid this problem a technique for using a cut down guide has been developed. Cut down guides are often used in wire localization biopsies. In such a procedure, a thin wire is inserted with a needle and the end of the wire positioned at the biopsy site. Typically, this is done in the x-ray department of a hospital or clinic. A small needle is inserted under x-ray guidance pointing toward the biopsy site. A wire and a hook on the end is passed through the needle and the hook is positioned at the biopsy site. The needle is removed and the wire left in place. The patient is then taken to an operating room where the surgeon uses the wire as a guide to reach the biopsy site.

A shortcoming to this needle insertion process in association with intraductal procedures is the additional invasion of a needle probe, as well as the need for additional x-rays, both of which create discomfort for the patient. It also often involves two different departments of a hospital further complicating matters. The present invention overcomes the shortcomings.

### SUMMARY OF THE INVENTION

An assembly suitable for use in mammary intraductal procedures as a biopsy site marker and cut down guide is provided. The assembly comprises an elongated filament that defines a hollow interior and has at least a pliable distal end portion together with a removable elongated core stiffener receivable within and guidable through the hollow interior. Alternatively, the entire filament may be pliable and formed of polypropylene, polyurethane, nylon or other extrudable pliant plastic material. The pliable distal end portion of the filament also includes at least one indicator suitable for marking a biopsy site. Preferably, the indicator is a radio-opaque marking on the filament. The core stiffener is formed of a rigid material, and is preferably formed of metal, such as a nickel-titanium allow, stainless or surgical steel, or the like. The core stiffener is guided through the hollow interior and provides a wire guide or cut down guide with which a physician or other practitioner is guided to the biopsy site.

The filament may be hollow along its entire length such that it takes the form of a tube. Alternatively, the filament may be hollow only along a portion of its length. For example, the distal end portion filament may include a solid portion. The distal end portion of the filament may also include an atraumatic distal end, such as a distal end portion that includes a taper, a rounded tip, or both. In such an embodiment, the filament may also be used as a dilator or obturator. Preferably, the filament also includes spaced penetration depth markers.

Unlike the prior methods described, the present assembly enables a practitioner to mark the biopsy site in an intraductal procedure with the filament, and leave the filament in place during the pathology of the obtained cellular or tissue sample, which can take several days. If it determined from the pathology that further tissue removal is required, such as if the sample proves malignant, the practitioner uses the filament as a guide for the core stiffener. The core stiffener is introduced and guided through the hollow interior of the filament. When the core stiffener is in place in the filament, the assembly acts as a cut down guide, such as the wire guide discussed above.

### BRIEF DESCRIPTION OF THE DRAWINGS

In the drawings,

FIG. 1 is a side schematic view of a preferred embodiment of an assembly embodying the present invention and suitable for use in mammary intraductal procedures as a biopsy site marker and cut down guide;

FIG. 2 is a perspective view of the filament of FIG. 1 inserted into a mammary duct, a portion of the human breast broken away to show interior detail;

FIG. 2A is an enlarged view of a portion of FIG. 2 showing the distal end portion of the filament at the biopsy site;

FIG. 3 is a perspective view of the filament of FIG. 2 with a core stiffener received in the hollow interior of the filament;

FIG. 4 is a perspective view of the assembly comprising the filament with core stiffener of FIG. 3 in use as a cut down guide;

FIG. 5 is a side schematic view of an alternate embodiment of an assembly suitable for use in mammary intraductal procedures as a biopsy site marker and cut down guide embodying the present invention wherein the filament is provided with an atraumatic distal end; and

FIG. 6 is a side schematic view of another preferred embodiment of an assembly embodying the present invention and suitable for use in mammary intraductal procedures as a biopsy site marker and cut down guide.

### DESCRIPTION OF THE PREFERRED EMBODIMENT OF THE INVENTION

The invention disclosed herein is, of course, susceptible of being embodied in many different devices. Shown in the drawings and described herein below in detail are preferred embodiments of the invention. It is to be understood, however, that the present disclosure is an exemplification of the principles of the invention and does not limit the invention to the illustrated embodiments.

An embodiment of the present assembly 10 is shown in FIG. 1. As shown, assembly 10 comprises a tubular filament 12 and a core stiffener 14 sized to be received therewithin. Disposed on the distal end portion 16 of the filament 12 is an indicator, such as radio-opaque marker 18. Filament 12 defines a hollow interior 20 and an open end 22. The hollow interior 20 extends along the entire length of filament 12 such that filament 12 forms a tube open at distal and proximal ends thereof. Also provided on the filament 12 are depth markers 24. Core stiffener 14 is sized to be slidably received within hollow interior 20.

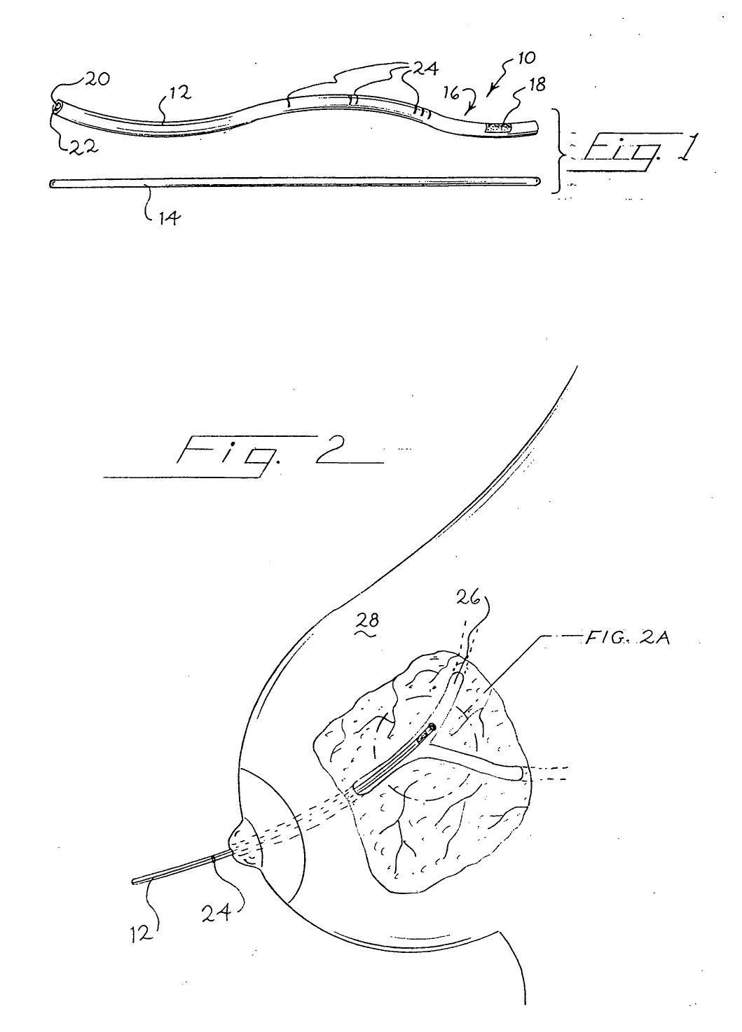

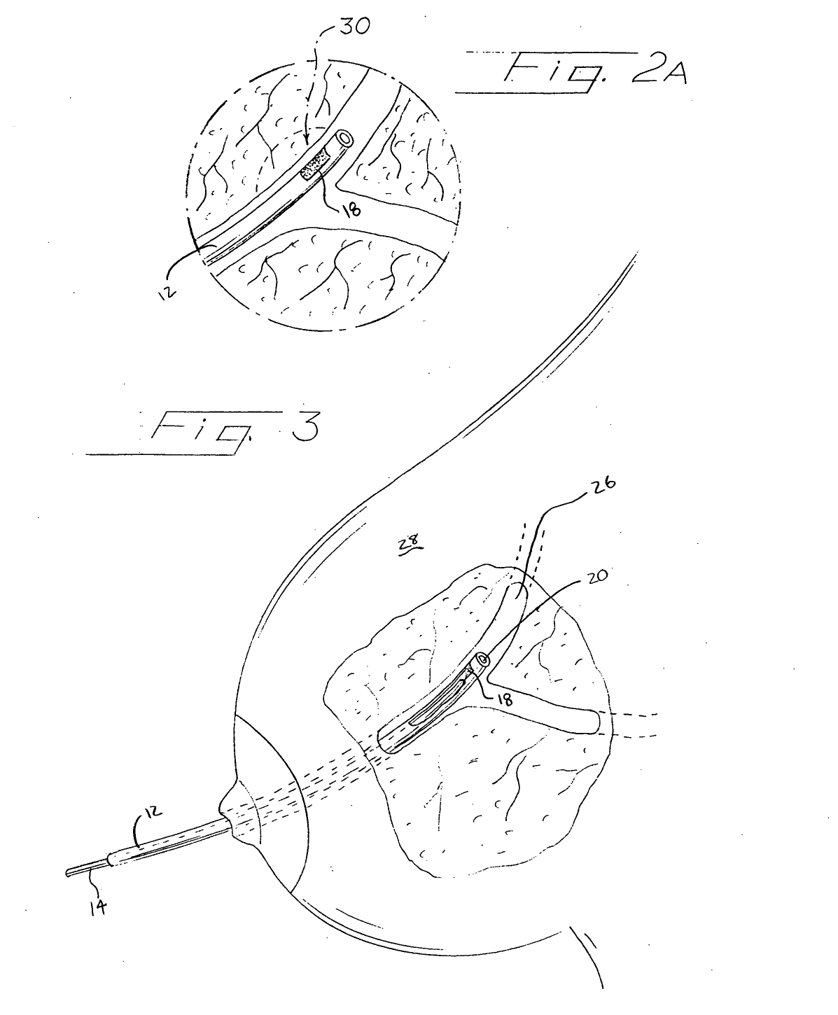

Referring to FIG. 2, the hollow filament 12 is shown inserted into a mammary duct 26 of a human breast 28. The distal end portion 16 of the filament 12 is guided along to mammary duct 26 until the radio-opaque marker 18 is positioned with the biopsy site 30 (FIG. 2A). To insert the filament, an introducer (not shown) is first guided into the mammary duct. Such an introducer also serves as the conduit for guiding other instruments, such as an endoscope, a biopsy device, and the like, into the mammary duct. In practice, the introducer is positioned at the biopsy site with the aid of an endoscope. After a biopsy is conducted, the biopsy device is removed and the filament 12 is inserted. The filament 12 may be left in the mammary duct 12 of the patient for several days while the results of the pathology are obtained. Excess length of the filament 12 can be cut away if desired. A surgical clip (not shown) can be secured to the filament to maintain its position within the patient until any later procedure.

To the extent it is determined from the pathology that a further biopsy is required, the core stiffener 14 may be inserted into hollow interior 20. Although the path of mammary ducts are not necessarily straight, typically the density of the breast tissue is such that it may be easily manipulated so that the core stiffener 16 can be guided through the hollow interior 20 as shown in FIG. 3. This is especially the case in older women where breast tissue has become less dense with age.

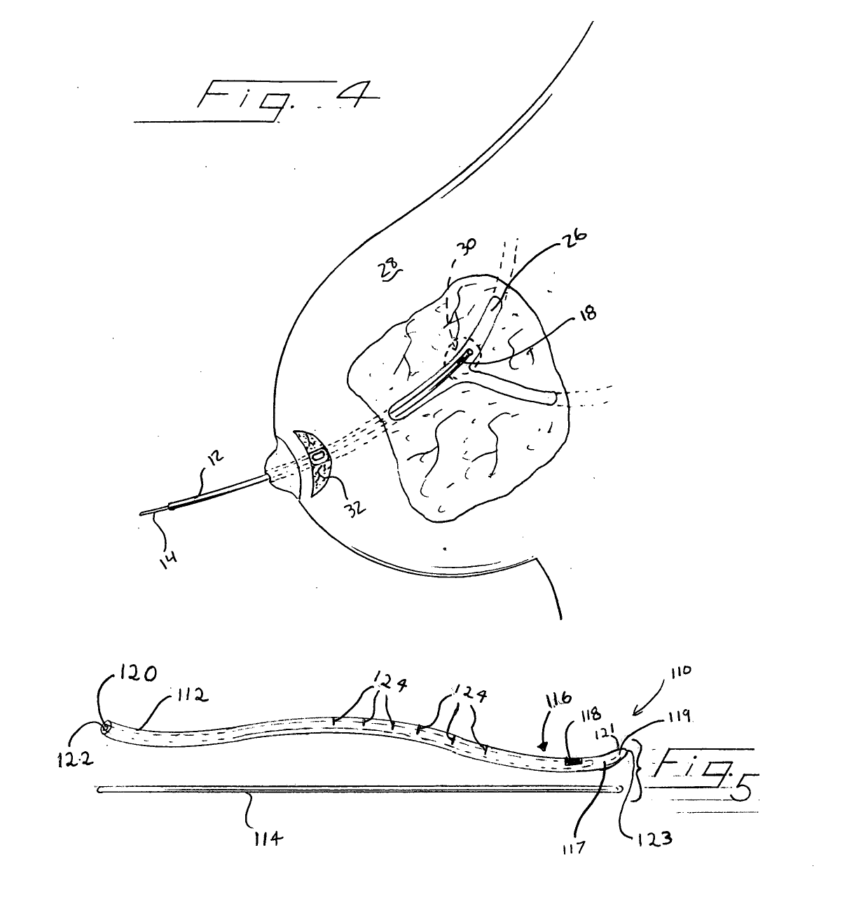

The assembly 10 thereby provides a cut down guide for a physician. As shown in FIG. 4, the assembly 10 is positioned within the patient's mammary duct with the indicator, such as radio-opaque marker 18, placed at the biopsy site 30. In this manner, a physician is able to make an incision, such as incision 32 and use the assembly 10 to guide him or her to the biopsy site as one would utilize a wire guide, but without the need for a separate needle insertion procedure. Because of the rigidity of the core stiffener 14, inadvertent contact by the physician's cautery or surgical knife will not cause the guide to be severed, whereas this would be a concern with only a filament. Further, because the assembly 10 is guided along the mammary duct, duct resection, where all or a portion of a duct is removed, is also made easier since the assembly, tracks the path along the target duct which typically bends and branches. By leaving the assembly 10 in place even after surgery, later investigation of the duct region can be performed to ensure that all the desired tissue has been removed.

The core stiffener 14 may also incorporate small amounts of radioactive material in the distal portion inserted into the filament 12, such as a radioactive coating or incorporation of radioactive particles into the core stiffener 14 itself. The radioactive material is selected to provide a low dose of radiation such that when the distal portion is left at the biopsy site 30 for a period of time, radiation therapy is provided to the desired region.

Another embodiment of the present invention is shown in FIG. 5. In this embodiment the assembly 110 again includes a filament 112 and a core stiffener 114. As with the previous embodiment, the distal end portion 116 of the filament 112 includes an indicator, such as radio-opaque marker 118. Filament 112 also defines a hollow interior 120 and an open end 122. In this embodiment, the hollow interior 120 does not extend along the entire length of filament 112 such that filament 112 forms a tube. Rather, the distal end portion 116 of the filament 112 includes a solid portion 117. An atraumatic tip 119 is also provided, and includes a taper 121 and a rounded tip 123. The atraumatic tip 119 is beneficial where the filament 112 is used to dilate or penetrate the nipple orifice to reach the mammary duct. As with the previous embodiment, the filament 112 also includes a plurality of depth markers 124.

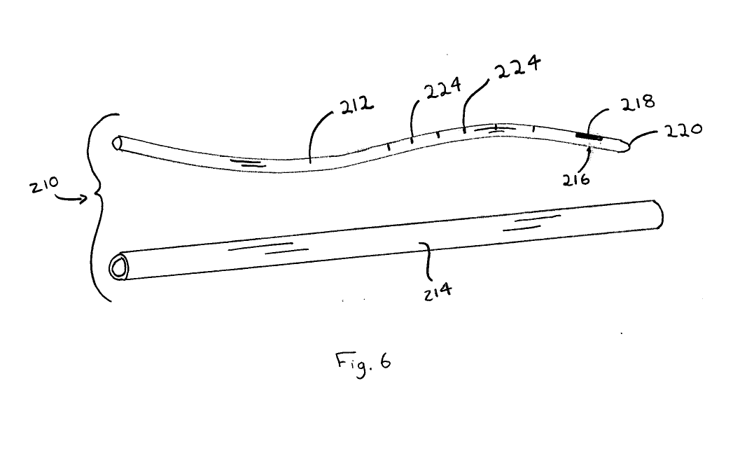

Another embodiment of the present assembly 210 is shown in FIG. 6. As shown, assembly 210 comprises a solid filament 212 and a hollow elongated stiffener 214 sized to be passed over and along the solid filament 212. An indicator, such as radio-opaque marker 218 is positioned on the distal end portion 216 of the filament 212. Preferably, the filament 212 includes an atraumatic tip. For example, the tip 220 may be rounded and include a taper. Also provided on the filament 212 are depth markers 224. In this embodiment, when the filament is inserted into the stiffener 214, the stiffener 214 covers the filament 212 such that during the cut-down operation, the filament 212 is not inadvertently severed.

As discussed above, the stiffener 214 can incorporate small amounts of radioactive material in the distal portion to provide a low dose of radiation such that when the distal portion is left at the biopsy site for a period of time, radiation therapy is provided to the desired region.

The foregoing description is to be taken as illustrative, but not limiting. Still other variants within the spirit and scope of the present invention will readily present themselves to those skilled in the art.

We claim:

1. A biopsy site marker and cut down guide assembly suitable for use in mammary intraductal procedures, the assembly comprising:
a filament, at least a portion of which defines a hollow interior and having a pliable distal end portion, the distal end portion of the filament further including at least one indicator; and
an insertable core stiffener sized to be slidably received within and guidable through the hollow interior of the filament.

2. The assembly of claim 1, wherein the indicator is a radio-opaque marker.

3. The assembly of claim 1, wherein the distal end portion of the filament is provided with an atraumatic distal end.

4. The assembly of claim 1, wherein the distal end portion of the filament further comprises a taper and a rounded tip.

5. The assembly of claim 1, wherein the filament further includes at least one penetration depth marker.

6. The assembly of claim 1, wherein the filament is pliable along the entire length thereof.

7. The assembly of claim 1, wherein the hollow interior extends through the entire length of the filament.

8. The assembly of claim 1, wherein the core stiffener includes radioactive material suitable for providing radioactive therapy to the biopsy site.

9. A method for marking a biopsy site and providing a cut down guide in a mammary intraductal procedure, the method comprising:
inserting a filament having a pliable distal end portion and an indicator by guiding the distal end portion thereof to the biopsy site;
positioning the indicator at the biopsy site; and
extending an elongated stiffener along the filament.

10. The method of claim 9, wherein the stiffener is a core stiffener and the filament is a hollow tube and the step of extending the elongated stiffener comprises inserting the stiffener into the filament and guiding the core stiffener along the hollow interior of the filament.

11. The method of claim 9, wherein the stiffener is a tubular stiffener and the step of extending the elongated stiffener comprises guiding the stiffener over the filament and guiding the stiffener along the exterior of the filament.

12. The method of claim 9, further comprising the step of removably securing the filament to the patient.

13. The method of claim 9, wherein the core stiffener includes radioactive material suitable for providing radioactive therapy to the biopsy site.

14. A biopsy site marker and cut down guide assembly suitable for use in mammary intraductal procedures, the assembly comprising:
a filament having a pliable distal end portion, the distal end portion of the filament further including at least one indicator; and
an elongated tubular stiffener sized to be slidably received over and guidable along the exterior of the filament.

15. The assembly of claim 14, wherein the indicator is a radio-opaque marker.

16. The assembly of claim 14, wherein the distal end portion of the filament is provided with an atraumatic distal end.

17. The assembly of claim 14, wherein the distal end portion of the filament further comprises a taper and a rounded tip.

18. The assembly of claim 14, wherein the filament further includes at least one penetration depth marker.

19. The assembly of claim 14, wherein the filament is pliable along the entire length thereof.

20. The assembly of claim 14, wherein the stiffener includes radioactive material suitable for providing radioactive therapy to the biopsy site.

21. A biopsy site marker and cut down guide assembly suitable for use in mammary intraductal procedures, the assembly comprising:
an elongated member having a pliable distal end portion suitable for dilating a nipple orifice of a mammary duct, the distal end portion of the filament further including at least one indicator that is detectable while the distal end portion of the filament is in the mammary duct; and
an elongated tubular stiffener insertable into the mammary duct and sized to be slidably received over and guidable along the exterior of the filament.

22. The assembly of claim 21, wherein the indicator is a radio-opaque marker.

23. The assembly of claim 21, wherein the distal end portion of the elongated member is provided with an atraumatic distal end.

24. The assembly of claim 21, wherein the distal end portion of the elongated member comprises a taper and a rounded tip.

25. The assembly of claim 21, wherein the elongated member further includes at least one penetration depth marker.

26. The assembly of claim 21, wherein the elongated member is pliable along the entire length thereof.

27. The assembly of claim 21, wherein the tubular stiffener includes radioactive material suitable for providing radioactive therapy to the biopsy site.
